(19) United States
(12) Patent Application Publication
(10) Pub. No.: US 2019/0216423 A1
(43) Pub. Date: Jul. 18, 2019

(54) ULTRASOUND IMAGING APPARATUS AND METHOD OF CONTROLLING THE SAME

(71) Applicant: SAMSUNG MEDISON CO., LTD., Hongcheon-gun (KR)

(72) Inventors: Jong Sun KO, Seoul (KR); Hae Kee MIN, Seongnam-si (KR); Seong Jin KIM, Hwaseong-si (KR)

(21) Appl. No.: 15/982,662

(22) Filed: May 17, 2018

(30) Foreign Application Priority Data
Jan. 18, 2018 (KR) 10-2018-0006346

(51) Int. Cl. A61B 8/08; A61B 8/14; A61B 8/00; A61B 17/34; A61B 34/20

(52) U.S. Cl. CPC A61B 8/0841; A61B 8/14; A61B 8/4455; A61B 10/0233; A61B 17/3403; A61B 34/20; A61B 8/463

(57) ABSTRACT

An ultrasound imaging apparatus accurately and efficiently diagnoses an object by providing cross-sectional images including a bioptic needle selected from images acquired by the ultrasound imaging apparatus and a guide line to guide motion of the bioptic needle and a method of controlling the ultrasound imaging apparatus. The ultrasound imaging apparatus comprises a display device; a probe configured to acquire an ultrasound image by emitting ultrasound to a surface of an object; and a controller configured to determine whether or not an image of a needle is comprised in at least one cross-sectional image constituting the ultrasound image of the object, and output the at least one cross-sectional image comprising the needle's image to the display device upon determination that the at least one cross-sectional image comprise the needle's image.

### CROSS-REFERENCE TO RELATED APPLICATION(S)

This application claims the benefit of Korean Patent Application No. 10-2018-0006346, filed on Jan. 18, 2018 in the Korean Intellectual Property Office, the disclosure of which is incorporated herein by reference.

### BACKGROUND

### 1. Field

Embodiments of the present disclosure relate to an ultrasound imaging apparatus to generate an image of the inside of an object by using ultrasound.

### 2. Description of the Related Art

An ultrasound imaging apparatus radiates ultrasonic signals toward a target region inside an object from a surface of the object and then collects reflected ultrasonic signals (ultrasonic echo signals) to non-invasively acquire tomograms of soft tissues or images of blood streams using information thereon. Ultrasound imaging apparatuses are relatively small in size and inexpensive, display an image in real time, and have high safety due to no radiation exposure as compared with other diagnostic imaging apparatuses such as X-ray diagnosis apparatuses, computerized tomography (CT) scanners, magnetic resonance imaging (MRI) apparatuses, and nuclear medicine diagnosis apparatuses. Thus, ultrasound imaging apparatuses have been widely used for heart diagnosis, celiac diagnosis, urinary diagnosis, and obstetric diagnosis.

An ultrasound imaging apparatus includes an ultrasound probe that transmits ultrasonic signals to an object and receives ultrasonic echo signals reflected by the object to acquire an ultrasound image of the object and a main body that generates an image of the inside of the object by using the ultrasonic echo signals received by the ultrasound probe.

Meanwhile, a user may treat a lesion inside a human body or collect a sample therefrom by using an ultrasound probe and a medical needle. In this case, the user needs to ascertain an accurate position of a needle for precise treatment and diagnosis. However, research on techniques of intuitively detecting the position of the needle is insufficient. Particularly, when a three-dimensional (3D) ultrasound image is acquired, it is difficult to ascertain the position of the needle, and there is a need to develop techniques of guiding the user to prevent penetration of a wrong position.

### SUMMARY

Therefore, it is an aspect of the present disclosure to provide an ultrasound imaging apparatus to accurately and efficiently diagnosing an object by providing cross-sectional images including a bioptic needle selected from images acquired by the ultrasound imaging apparatus and a guide line to guide motion of the bioptic needle and a method of controlling the ultrasound imaging apparatus.

Additional aspects of the disclosure will be set forth in part in the description which follows and, in part, will be obvious from the description, or may be learned by practice of the disclosure.

In accordance with one aspect of the present disclosure, an ultrasound imaging apparatus comprising: a display device; a probe configured to acquire an ultrasound image by emitting ultrasound to a surface of an object; and a controller configured to determine whether or not an image of a needle is comprised in at least one cross-sectional image constituting the ultrasound image of the object, and output the at least one cross-sectional image comprising the needle's image to the display device upon determination that the at least one cross-sectional image comprise the needle's image.

The controller may output the at least one cross-sectional image comprising the needle's image corresponding to a changed position of the needle to the display device when a position of the needle is changed

The controller may output the at least one cross-sectional image comprising the needle's image to the display device in real time.

The controller may generate a guide line from an insertion point of the needle to a predetermined target point and outputs the guide line to the display device when the needle is inserted into the object,

The controller may derive a difference between the needle's image and the guide line, generate a guide marker based on a position of the guide line by using the needle's image as a reference, and output the guide marker to the display device.

The controller may derive a guide marker based on a relationship between an extended line of the needle's image comprised in the at least one cross-sectional image and the position of the predetermined target point, and outputs the guide marker to the display device.

The controller may track a position of the needle's image comprised in the at least one cross-sectional image in real time and outputs the at least one cross-sectional image corresponding to the needle's image to the display device.

The controller may derive a predicted position of the needle after a current point of time based on positions of the needle's image from a point of time in the past to the current point of time, derive the at least one cross-sectional image comprising the predicted position of the needle, and output the at least one cross-sectional image to the display device.

The controller may control the ultrasound image of the object comprising at least one cross-sectional image comprising the needle's image to be output to the display device when the needle is inserted into the object.

In accordance with one aspect of the present disclosure, an ultrasound imaging apparatus may further comprise a sensing device configured to acquire position information of the needle.

The controller may derive the needle's image comprised in the at least one cross-sectional image based on the position information of the needle.

The ultrasound probe may comprise at least one of a matrix probe and a three-dimensional (3D) probe.

In accordance with one aspect of the present disclosure, a method of controlling an ultrasound imaging apparatus, the method comprising: acquiring an ultrasound image by emitting ultrasound to a surface of an object, determining whether or not an image of a needle is comprised in at least one cross-sectional image constituting the ultrasound image of the ultrasound image, and outputting the at least one cross-sectional image comprising the needle's image to a display device upon determination that the needle's image is comprised in at least one cross-section image.

In accordance with one aspect of the present disclosure, the method may further comprise outputting the at least one cross-sectional image comprising the needle's image corresponding to a changed position of the needle to the display device when a position of the needle is changed.

The outputting of the at least one cross-sectional image may comprise outputting the at least one cross-sectional image comprising the needle's image in real time.

In accordance with one aspect of the present disclosure, the method may further comprise generating a guide line from an insertion point of the needle to a predetermined target point when the needle is inserted into the object.

In accordance with one aspect of the present disclosure, the method may further comprise deriving a difference between the needle's image and the guide line, and generating a guide marker based on a position of the guide line by using the needle's image as a reference.

The outputting of the at least one cross-sectional image may further comprise outputting the guide marker to the display device.

The generating of a guide marker may comprise deriving a guide marker based on a relationship between an extended line of the needle's image comprised in the at least one cross-sectional image and a predetermined target point.

In accordance with one aspect of the present disclosure, the method may further comprise tracking a position of the needle's image comprised in the at least one cross-sectional image in real time.

In accordance with one aspect of the present disclosure, further comprise deriving a predicted position of the needle after a current point of time based on positions of the needle's image from a point of time in the past to the current point of time, and deriving the at least one cross-sectional image comprising the predicted position of the needle.

The outputting of the at least one cross-sectional image further comprises outputting an ultrasound image of the object comprising at least one cross-sectional image having the needle's image when the needle is inserted into the object.

In accordance with one aspect of the present disclosure, further comprise acquiring position information of the needle,

The outputting of the at least one cross-sectional image may further comprise outputting the needle's image comprised in the at least one cross-sectional image based on the position information of the needle.

The ultrasound probe may comprises at least one of a matrix probe and a three-dimensional (3D) probe.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a perspective view of an ultrasound imaging apparatus according to an embodiment.

FIG. 2 is a control block diagram of the ultrasound imaging apparatus.

FIG. 3 is a control block diagram illustrating the configuration of a main body of the ultrasound imaging apparatus in detail.

FIG. 4 is a control block diagram schematically illustrating the configuration of a main body of an ultrasound imaging apparatus according to an embodiment.

FIG. 5 is a diagram illustrating a needle N and an ultrasound probe according to the disclosed embodiment.

FIGS. 6A and 6B are diagrams illustrating methods of acquiring cross-sectional images constituting an ultrasound image according to an embodiment.

FIG. 7 is a diagram illustrating a cross-sectional image of an ultrasound image including an image I of a needle N according to an embodiment.

FIGS. 8A and 8B are diagrams illustrating an image 11 of the needle N at an initial time point according to an embodiment.

FIGS. 9A and 9B are diagrams illustrating a guide line and a guide marker to guide the needle N according to an embodiment.

FIGS. 10 and 11 are diagrams illustrating operations of outputting an ultrasound image and an image to guide the position of the needle N according to an embodiment.

FIGS. 12 to 14 are flowcharts according to an embodiment.

### DETAILED DESCRIPTION

Reference will now be made in detail to the embodiments of the present disclosure, examples of which are illustrated in the accompanying drawings, wherein like reference numerals refer to like elements throughout. This specification does not describe all elements of the embodiments of the present disclosure and detailed descriptions on what are well known in the art or redundant descriptions on substantially the same configurations may be omitted. The terms ‘unit’, ‘module’, ‘member’, or ‘block’ used in the specification may be implemented using a software or hardware component. According to an embodiment, a plurality of ‘units’, ‘modules’, ‘members’, or ‘blocks’ may also be implemented using an element and one ‘unit’, ‘module’, ‘member’, or ‘block’ may include a plurality of elements

Throughout the specification, when an element is referred to as being ‘connected to’ another element, it may be directly or indirectly connected to the other element and the ‘indirectly connected to’ includes connected to the other element via a wireless communication network.

Also, it is to be understood that the terms ‘include’ or ‘have’ are intended to indicate the existence of elements disclosed in the specification, and are not intended to preclude the possibility that one or more other elements may exist or may be added.

Throughout the specification, it will be understood that when one element, is referred to as being ‘on’ another element, it can be directly on the other element, or intervening elements may also be present therebetween.

The terms ‘first’, ‘second’, etc. are used to distinguish one component from other components and, therefore, the components are not limited by the terms.

An expression used in the singular encompasses the expression of the plural, unless it has a clearly different meaning in the context.

The reference numerals used in operations are used for descriptive convenience and are not intended to describe the order of operations and the operations may be performed in a different order unless otherwise stated.

Hereinafter, operating principles and embodiments of the present disclosure will be described with reference to the accompanying drawings.

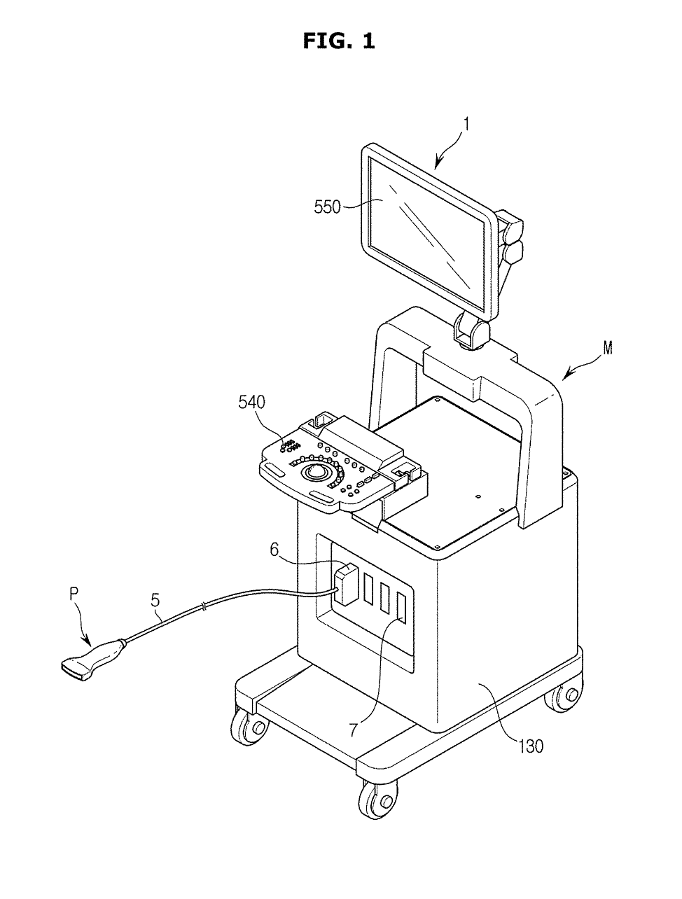

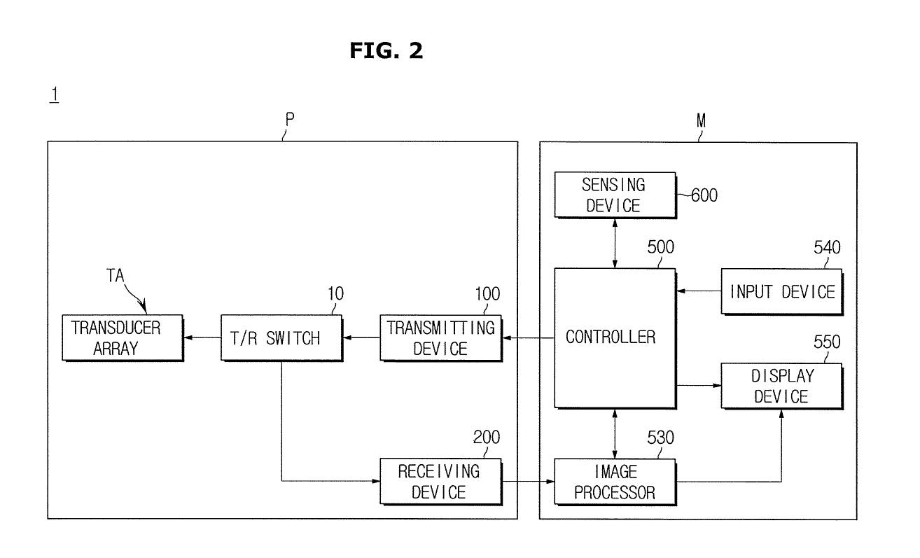

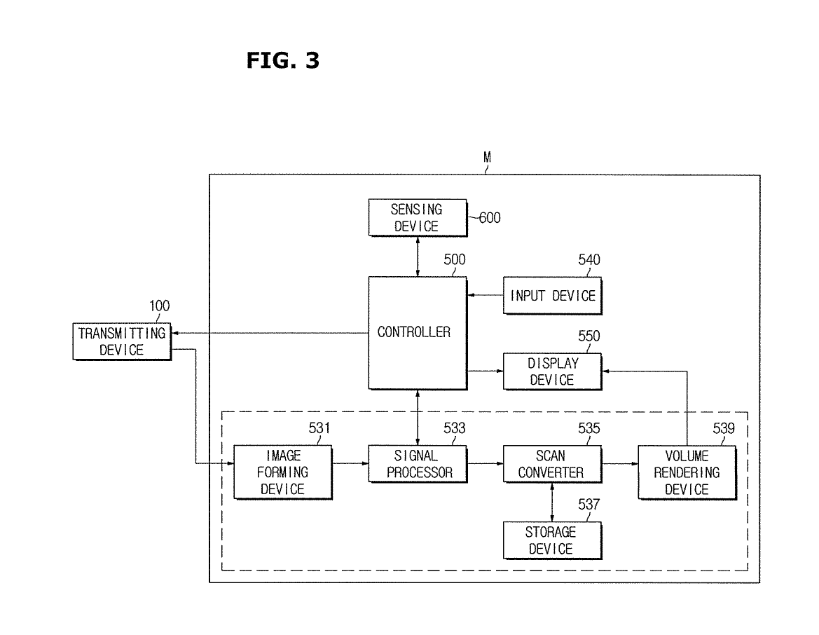

FIG. 1 is a perspective view of an ultrasound imaging apparatus according to an embodiment. FIG. 2 is a control block diagram of the ultrasound imaging apparatus. FIG. 3 is a control block diagram illustrating the configuration of a main body of the ultrasound imaging apparatus in detail.

Referring to FIG. 1, an ultrasound imaging apparatus 1 includes an ultrasound probe P configured to transmit ultrasonic signals to an object, receive ultrasonic echo signals from the object, and converting the received signals into electric signals, and a main body M connected to the ultrasound probe P, including an input device 540 and a display device 550, and displaying an ultrasound image. The ultrasound probe P may be connected to the main body M of the ultrasound imaging apparatus via a cable 5 to receive various signals required to control the ultrasound probe P or transmit analog signals or digital signals corresponding to the ultrasonic echo signals received by the ultrasound probe P to the main body M. However, examples of the ultrasound probe P are not limited thereto and the ultrasound probe P may also be implemented using a wireless probe capable of transmitting/receiving signals to/from the main body M via a network formed between the ultrasound probe P and the main body M.

One end of the cable 5 may be connected to the ultrasound probe P and the other end may be provided with a connector 6 to be coupled to or separated from a slot 7 of the main body M. The main body M and the ultrasound probe P may exchange control commands or data by using the cable 5. For example, when the user inputs information on depth of focus, size or shape of aperture, steering angle, or the like via the input device 540, the information may be transmitted to the ultrasound probe P via the cable 5 and used for transmit/receive beamforming of a transmitting device 100 and a receiving device 200. Alternatively, when the ultrasound probe P is implemented using a wireless probe as described above, the ultrasound probe P is connected to the main body M via a wireless network instead of the cable 5. In the case where the ultrasound probe P is connected to the main body M via the wireless network, the main body M and the ultrasound probe P may also exchange the control commands or data described above. As illustrated in FIG. 2, the main body M may include a controller 500, an image processor 530, an input device 540, and a display device 550.

The controller 500 controls the overall operation of the ultrasound imaging apparatus 1. Specifically, the controller 500 controls the operation of each of the components by generating control signals to control the components of the ultrasound imaging apparatus 1 respectively, e.g., the transmitting device 100, a T/R switch 10, the receiving device 200, the image processor 530, the display device 550, and the like illustrated in FIG. 2. In the ultrasound imaging apparatus 1 illustrated in FIGS. 2 and 3, a transmit/receive beamformer is included not in the main body M but in the ultrasound probe P. However, the transmit/receive beamformer may also be included in the main body M rather than the ultrasound probe P.

The controller 500 calculates delay profiles of a plurality of ultrasound transducer elements 60 constituting an ultrasound transducer array TA and calculates time delay values in accordance with differences between each of the plurality of ultrasound transducer elements 60 included in the ultrasound transducer array TA and a focal point of the object based on the calculated delay profiles. In addition, the controller 500 controls the transmit/receive beamformer in accordance therewith to generate transmit/receive signals.

Also, the controller 500 may control the ultrasound imaging apparatus 1 by generating control commands for the respective components of the ultrasound imaging apparatus 1 in accordance with instructions or commands of the user input via the input device 540.

The image processor 530 generates an ultrasound image of a target region inside the object based on ultrasonic signals focused by the receiving device 200.

Referring to FIG. 3, the image processor 530 may include an image forming device 531, a signal processor 533, a scan converter 535, a storage device 537, and a volume rendering device 539.

The image forming device 531 generates a coherent two-dimensional (2D) or three-dimensional (3D) image of the target region inside the object based on ultrasonic signals received by the receiving device 200.

The signal processor 533 converts information on the coherent image generated by the image forming device 531 into ultrasound image information according to a diagnosis mode such as a brightness mode (B-mode) and a Doppler mode (D-mode). For example, when the diagnosis mode is set to the B-mode, the signal processor 533 performs analog/digital (ND) conversion, or the like and generates ultrasound image information for a B-mode image in real time. Alternatively, when the diagnosis mode is set to the D-mode, the signal processor 533 extracts information on phase changes from the ultrasonic signal, calculates information on a blood stream corresponding to each point of cross-sectional image such as speed, power, and distribution, and generates ultrasound image information for a D-mode image in real time.

The scan converter 535 converts the converted ultrasound image information received from the signal processor 533 and the converted ultrasound image information stored in the storage device 537 into general video signals for the display device 550 and transmits the converted signals to the volume rendering device 539.

The storage device 537 temporarily or non-temporarily stores the ultrasound image information converted by the signal processor 533.

The volume rendering device 539 performs volume rendering based on the video signals received from the scan converter 535, corrects rendered image information to generate a final resultant image, and transmits the generated resultant image to the display device 550.

The input device 540 allows the user to input a command related to the operation of the ultrasound imaging apparatus 1. The user may input or set an ultrasound diagnosis start command, a diagnosis mode select command to select the B-mode, a motion mode (M-mode), the D-mode, an elastography mode (E-mode), or a 3D-mode, region of interest (ROI) setting information including size and position of a ROI, and the like via the input device 540.

A B-mode image refers to an image displaying the cross-section of the inside of the object and portions with strong echo signals are distinguished from portions with weak echo signals by modulating brightness. The B-mode image is generated based on information obtained from tens to hundreds of scan lines.

An M-mode refers to an image representing changes over time in biometric information (e.g., brightness information) on a particular portion (M line) in a cross-sectional image (B-mode image). In general, the B-mode image and the M-mode image are simultaneously displayed on one screen to allow to the user to accurately diagnose by comparing and analyzing the two types of data.

A D-mode image refers to an image of a moving object obtained by the Doppler effect in which a frequency of sound emitted from a moving object changes. Modes using the Doppler effect may further be classified into a power Doppler imaging (PDI) mode, a color flow (S Flow) mode, and a directional power Doppler imaging (DPDI) mode.

A PDI mode image refers to an image representing the degree of Doppler signal or the number of structures (number of erythrocytes in blood). In the PDI mode, there is no aliasing signals due to less sensitivity to an angle of incidence and image attenuation caused by noise decreases. Also, since reflected Doppler energy is recorded, the PDI mode is very sensitive enabling detection of small blood vessels and blood streams with low speed.

The S Flow mode provides a power image (PDI) representing the power of a Doppler signal in 2D distribution and a velocity image representing the velocity of the Doppler signal in 2D distribution. A S flow image may not only visualize blood streams in real time but also represent a wide range of blood stream statuses from a high velocity blood stream in a larger blood vessel to a low velocity blood stream in a smaller blood vessel.

A DPDI mode image refers to a directional image representing information on a direction of a Doppler signal in 2D distribution in the PDI mode. Thus, the DPDI mode may detect information on blood streams more accurately than the PDI mode. In addition, an M-mode image may be generated in the D-mode.

The E-mode refers to a method of acquiring an ultrasound elastography image by using elastography. In this regard, elastography refers to an analysis of a phenomenon in which elasticity of tissues decreases in a hard structure such as malignant mass, and thus the degree of deformation of the tissues by pressure decreases. An ultrasound elastography image refers to an image quantitatively representing stiffness of tissues. Particularly, the E-mode has been widely used in diagnosis of cervix cancer, breast cancer, or prostate cancer.

A 3D-mode image refers to an image representing a geometric conformation or a space including X, Y, and Z values respectively representing depth, width, and height or a series of images indicating a stereoscopic feeling as a 3D shape or providing a stereoscopic effect. For example, the user may display a face shape of a fetus by using stereoscopic effects of the 3D-mode and provide parents of the fetus with the face shape.

The input device 540 may include various devices allowing the user to input data, instructions, and commands, such as a keyboard, a mouse, a trackball, a tablet, or a touch screen module.

The display device 550 displays a menu or information required for ultrasound diagnosis, an ultrasound image acquired during an ultrasound diagnosis process, and the like. The display device 550 displays an ultrasound image of a target region inside the object generated by the image processor 530. The ultrasound image displayed on the display device 550 may be a B-mode ultrasound image, an E-mode ultrasound image, or a 3D ultrasound image. The display device 550 may display various ultrasound images obtained according to the afore-mentioned modes.

The display device 550 may be implemented using various known displays such as a cathode ray tube (CRT) and a liquid crystal display (LCD).

The ultrasound probe P according to an embodiment may include the transducer array TA, the T/R switch 10, the transmitting device 100, and the receiving device 200 as illustrated in FIG. 2. The transducer array TA is provided at one end of the ultrasound probe P. The ultrasound transducer array TA refers to a one-dimensional (1D) or 2D array of a plurality of ultrasound transducer elements 60. While the ultrasound transducer array TA oscillates by pulse signals or alternating currents supplied thereto, ultrasound is generated. The generated ultrasound is transmitted to the target region inside the object. In this case, the ultrasound generated by the ultrasound transducer array TA may also be transmitted to a plurality of target regions inside the object. In other words, the generated ultrasound may be multi-focused and transmitted to the plurality of target regions.

The ultrasound generated by the ultrasound transducer array TA may be reflected by the target region inside the object and then return to the ultrasound transducer array TA. The ultrasound transducer array TA receives ultrasonic echo signals returning after being reflected by the target region. When an ultrasonic echo signal arrives at the ultrasound transducer array TA, the ultrasound transducer array TA oscillates at a predetermined frequency corresponding to a frequency of the ultrasonic echo signal and outputs an alternating current having a frequency corresponding to the oscillation frequency. Thus, the ultrasound transducer array TA may convert the received ultrasonic echo signal into an electric signal. Since each of the ultrasound transducer elements 60 outputs an electric signal by receiving the ultrasonic echo signal, the ultrasound transducer array TA may output electric signals of a plurality of channels.

The ultrasound transducer may be implemented using a magnetostrictive ultrasonic transducer using a magnetostrictive effect of a magnetic material, a piezoelectric ultrasonic transducer using a piezoelectric effect of a piezoelectric material, or a capacitive micromachined ultrasonic transducer (cMUT) that receives ultrasound using oscillation of hundreds or thousands of micromachined thin films. In addition, any other types of transducers capable of generating ultrasound in accordance with electric signals or generating electric signals in accordance with ultrasound may also be used as the ultrasound transducer.

For example, the transducer elements 60 according to an embodiment may include a piezoelectric vibrator or a thin film. When an alternating current is supplied from a power source, the piezoelectric vibrator or the thin film vibrates at a predetermined frequency in accordance with the supplied alternating current and generates ultrasound having the predetermined frequency in accordance with the vibration frequency. On the contrary, when an ultrasonic echo signal having a predetermined frequency arrives at the piezoelectric vibrator or the thin film, the piezoelectric vibrator or the thin film vibrates in accordance with the ultrasonic echo signal and outputs an alternating current of a frequency corresponding to the vibration frequency.

The transmitting device 100 applies transmit purses to the transducer array TA to control the transducer array TA to transmit ultrasonic signals to the target region inside the object. The transmitting device may include a transmit beamformer and a pulser.

The transmit beamformer 110 generates a transmit signal pattern in accordance with a control signal of the controller 500 of the main body M and outputs the transmit signal pattern to a pulser 120. The transmit beamformer 110 generates the transmit signal pattern based on a time delay value of each of the ultrasound transducer elements 60 constituting the transducer array TA calculated by the controller 500 and transmits the generated transmit signal pattern to the pulser 120.

The receiving device 200 performs a predetermined processing on ultrasonic echo signals received by the transducer array TA and performs receive beamforming. The receiving device 200 may include a receive signal processor and a receive beamformer. The electric signals converted by the transducer array TA are input to the receive signal processor. The receive signal processor may amplify the electric signals converted from the ultrasonic echo signals before processing the electric signals or performing time delay processing on the electric signals and may adjust gains or compensate attenuation according to depth. More particularly, the receive signal processor may include a low noise amplifier (LNA) to reduce noise of the electric signals received from the ultrasound transducer array TA and a variable gain amplifier (VGA) to control gain values in accordance with the input signals. The VGA may be, but is not limited to, a time gain compensator (TGC) to compensate gains in accordance with distance from the focal point.

The receive beamformer performs beamforming for the electric signals received from the receive signal processor. The receive beamformer increases intensities of the signals received from the receive signal processor through superposition. The electric signals beamformed by the receive beamformer are converted into digital signals by an A/D converter and transmitted to the image processor 530 of the main body M. When the main body M includes the ND converter, analog signals beamformed by the receive beamformer may also be transmitted to the main body M and converted into digital signals in the main body M. Alternatively, the receive beamformer may be a digital beamformer. The digital beamformer may include a storage device to sample analog signals and store the sampled signals, a sampling period controller 500 to control a sampling period, an amplifier to adjust a sample size, an anti-aliasing low pass filter to prevent aliasing before sampling, a bandpass filter to select a desired frequency band, an interpolation filter to increase a sampling rate while performing beamforming, a high-pass filter to remove a direct current (DC) component or a low frequency band signal, and the like.

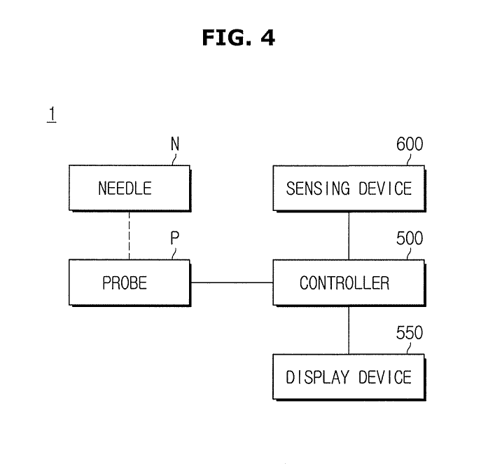

FIG. 4 is a control block diagram schematically illustrating the configuration of a main body of an ultrasound imaging apparatus according to an embodiment.

Referring to FIG. 4, the ultrasound imaging apparatus includes a needle N, a probe, a display device 550, and a controller 500.

The needle N may be a needle for biopsy to treat a lesion inside a human body or collect a sample therefrom. The needle N may be used in a state of being attached to the probe or separated therefrom.

The controller 500 may determine whether or not an image of the needle N is included in at least one cross-sectional image constituting the ultrasound image of the object by using position information of the needle N and output the at least one cross-sectional image to the display device 550 upon determination that the needle's image is included in the at least one cross-sectional image.

The ultrasound image may include at least one cross-sectional image of the object. According to an embodiment, the controller 500 may determine whether or not a cross-sectional image includes the needle's image based on position information of the needle N and feature points of the needle N.

The controller 500 may output the at least one cross-sectional image including the needle's image to the display device 550 in real time based on the position information of the needle N.

When the needle N is inserted into the object, the controller 500 may generate a guide line from an insertion point of the needle N to a predetermined target point and output the guide line to the display device 550. When the user aims at making the needle N to reach the lesion inside the object, the user may set a lesion as a target point. In addition, when the user uses the needle N for an invasive procedure, the controller 500 may generate a guide line from a start point of the invasive procedure to a position of the lesion. A method of generating the guide line will be described in detail later.

The controller 500 may derive a difference between the position of the needle N and the guide line and generate a guide marker based on the position of the guide line by using the position of the needle N as a reference.

When the guide line and the needle N do not coincide with each other, the guide marker may be implemented as a marker for indicating a direction to align the guide line and the needle N. The guide marker may be implemented using an arrow.

Also, the controller 500 may track the position of the needle N in real time and output the position of the tracked needle N to the display device 550.

Meanwhile, the controller 500 may derive information on a predicted position of the needle N after a current point of time based on position information of the needle N from a point of time in the past to the current point of time.

The controller 500 may also derive at least one cross-sectional image including the information on the predicted position of the needle N.

When the needle N is inserted into the object, the controller 500 may control an ultrasound image of the object including the at least one cross-sectional image having the needle N to be displayed in the display device 550. While the controller 500 outputs an ultrasound image to the display device 550 during a normal diagnosis, the controller 500 may change an existing image with an image including the needle N when the needle N is inserted into the object.

In addition, since the at least one cross-sectional image of the ultrasound image is required to constitute a stereoscopic image, the ultrasound probe may include at least one of a matrix probe and a 3D probe.

The controller 500 may be implemented using a memory (not shown) that stores data on algorithms to control the operation of components of the ultrasound imaging apparatus or programs to run the algorithms and a processor (not shown) that performs the aforementioned operation by using data stored in the memory. In this case, the memory and the processor may be implemented as separate chips. Alternatively, the memory and the processor may be implemented as a single chip.

Meanwhile, the ultrasound imaging apparatus may further include a sensing device to acquire position information of the needle N. The needle N may be magnetized. The sensing device may include a magnetic sensor and derive position information of the needle N by detecting a magnetic field changing in accordance with the position of the needle N. The position information refers to information based on an actual position of the needle N and may be any information enabling determination of the position of the needle N by using a magnetic field without limitation.

At least one component may be added or deleted corresponding to performance of the components of the ultrasound imaging apparatus illustrated in

FIGS. 2 to 4. In addition, it will be readily understood by those skilled in the art that mutual positions of the components may be changed to correspond to performance or structure of a system.

Meanwhile, each of the components illustrated in FIGS. 2 to 4 may be a software and/or hardware component such as a Field Programmable Gate Array (FPGA) and an Application Specific Integrated Circuit (ASIC).

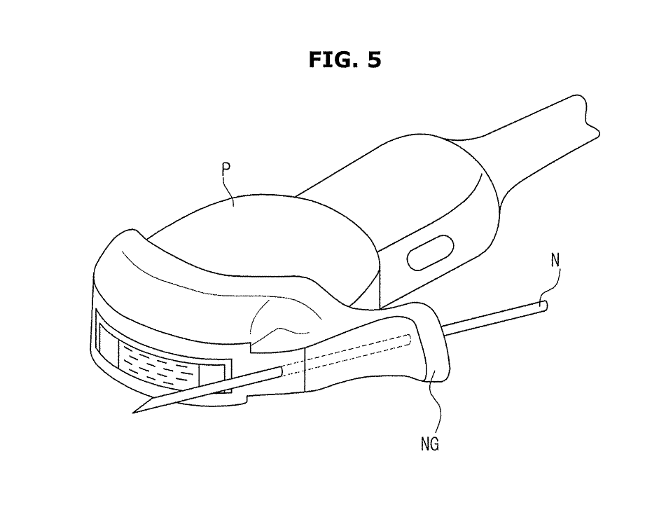

FIG. 5 is a diagram illustrating a needle N and an ultrasound probe according to the disclosed embodiment.

FIG. 5 illustrates an example in which a bioptic needle N is coupled to a needle guide NG installed in a main probe. The needle N may be coupled to the needle guide NG.

A diameter of the needle N may be smaller than a diameter of a guide channel. As illustrated in FIG. 5, the needle N may enter an inlet of a needle guide 112, move along the guide channel, and protrude out of the needle guide 112 through an outlet. The coupled bioptic needle N is inserted into the inside of the object and may collect a sample of a tissue of a region of interest.

FIG. 5 illustrates a configuration in which the needle N is coupled to the probe P. The needle N may be implemented in an attached form to the ultrasound probe P as illustrated in FIG. 5 but may also be implemented in a separated form from the ultrasound probe P.

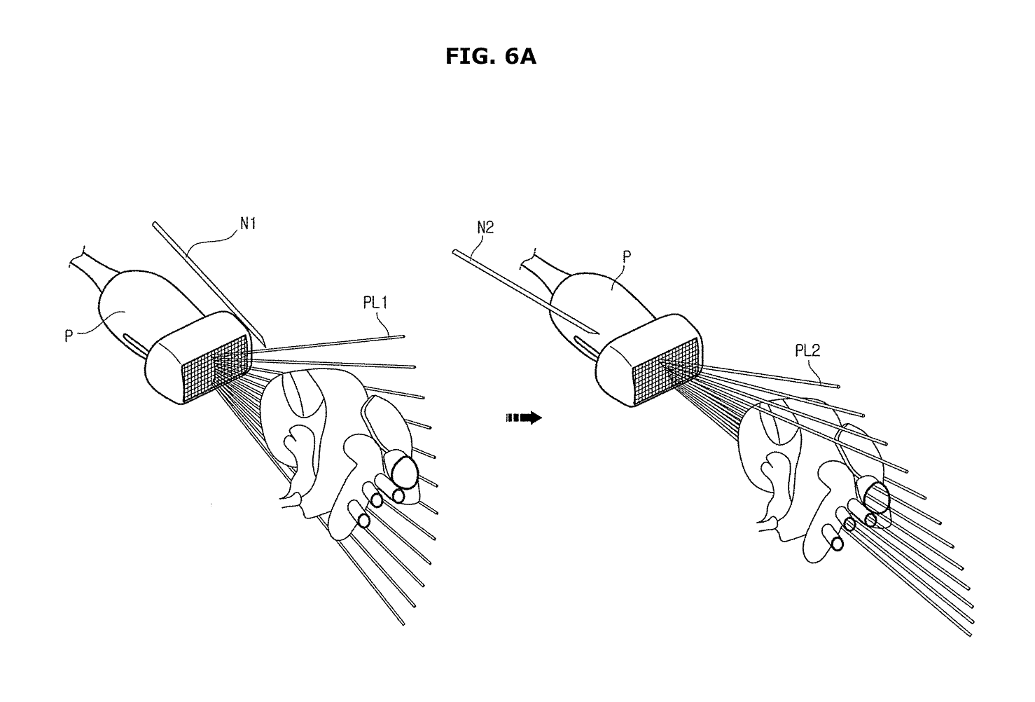

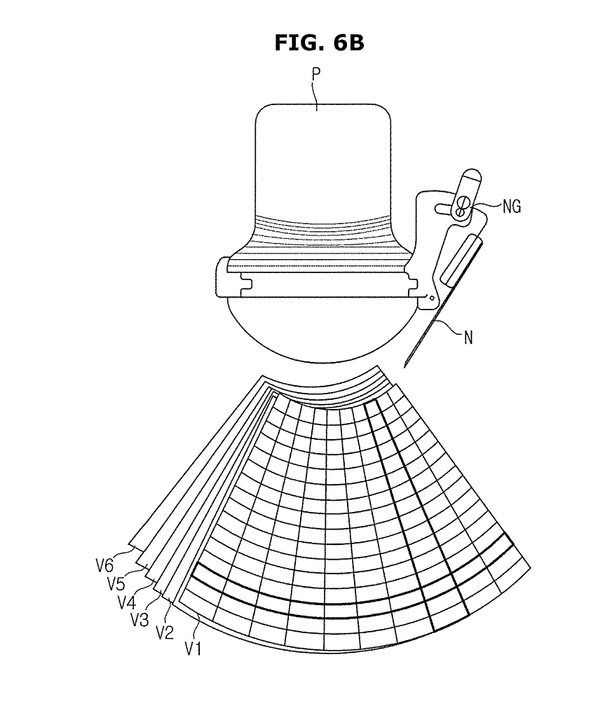

FIGS. 6A and 6B are diagrams illustrating methods of acquiring cross-sectional images constituting an ultrasound image according to an embodiment.

Referring to FIG. 6A an ultrasound probe P, a needle N, and an object are illustrated.

The probe P may be provided as a matrix probe. The matrix probe may acquire a 3D volume image. Also, the probe P may acquire volume data of a space. Thus, the user may generate one or more desired cross-sectional images PL1 and PL2. An angle of a cross-sectional image generated by the controller is not limited.

In addition, FIG. 6A illustrates that one cross-section of an ultrasound image coincides with the needle N. That is, the controller may identify at least one cross-sectional image PL1 and PL2 including a needle's image and acquire, in real time, cross-sectional images PL1 and PL2 of an ultrasound image including the needle's image by tracking the position of the needle N in real time. That is, when the position of the needle N is changed, the controller may derive the at least one cross-sectional image PL1 and PL2 including the needle's image corresponding to a changed position of the needle N.

Specifically, FIG. 6A illustrates a case in which the position of the needle N is changed from an initial position N1 to a later position N2. Although types of position change are not limited, FIG. 6A exemplarily illustrates a case in which the needle N rotates. In this case, the controller may select a cross-sectional image PL2 corresponding to the position of the needle N among cross-sectional images constituting the ultrasound image. According to an embodiment, the cross-section may be determined as the cross-section PL2 having rotated to correspond to rotation of the needle N. Although the case in which the needle N rotates is described with reference to FIG. 6A, the position change of the needle N is not limited to rotation.

Referring to FIG. 6B, methods of acquiring a stereoscopic image and a cross-sectional image based on signals acquired by the ultrasound probe are illustrated. The cross-sectional images of the stereoscopic image may be generated in a process of generating images based on the signals acquired by the ultrasound probe and an ultrasound image may be formed as a set of the cross-sectional images. The controller 500 may also identify an image including the needle N among the cross-sectional image of the stereoscopic image. When the controller 500 determines whether or not a cross-sectional image includes the needle, position information of the needle N acquired by the sensing device or information on feature points of the needle N included in the image may be used. For example, when a cross-sectional image V3 includes the needle among a plurality of cross-sectional images V1 to V6, the controller 500 may control the cross-sectional image V3 to be output to the display device 550.

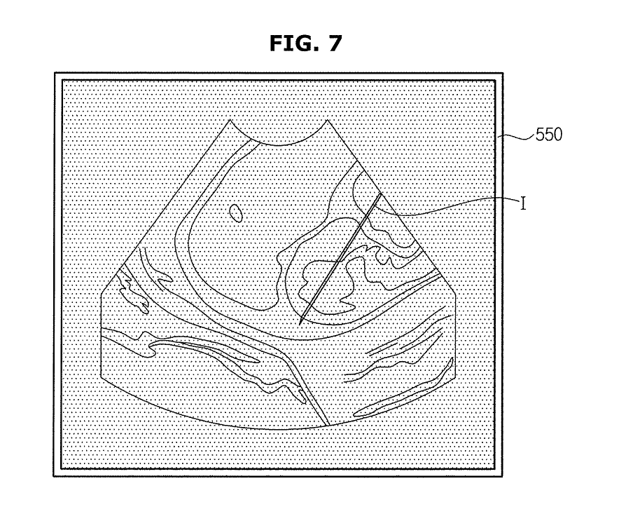

FIG. 7 is a diagram illustrating a cross-sectional image of an ultrasound image including an image I of a needle N according to an embodiment.

Referring to FIG. 7, among at least one cross-sectional image constituting the ultrasound image, a cross-sectional image including an image I of the needle N is shown as described above with reference to FIGS. 6A and 6B. Referring to FIG. 6B (?), the plurality of cross-sectional images V1 to V6 are derived and the cross-sectional image V3 including the image I of the needle N selected therefrom. The image illustrated in FIG. 7 may be implemented as the selected image.

Meanwhile, although there may be a plurality of cross-sectional images including the needle N, the controller 500 may output an image optimized to detect the position of the needle N to the display 550 in consideration of the position of the needle N and feature points of the needle N. Also, the controller 500 may identify a plurality of cross-sectional images to observe the position of the needle N and output the cross-sectional images to the display device 550.

Meanwhile, although FIGS. 6A, 6B, and 7 illustrate operations of outputting the cross-sectional images according to an embodiment, the operations are not limited thereto so long as the position of the needle N may be identified by converting volumes scanned by the ultrasound probe into cross-sectional images.

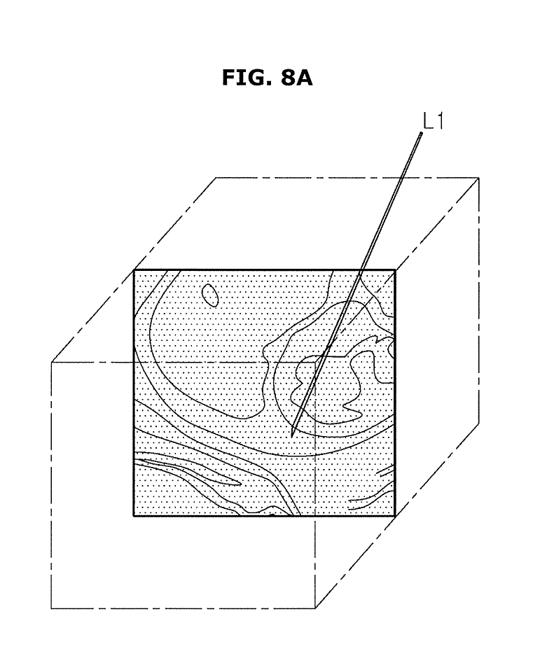

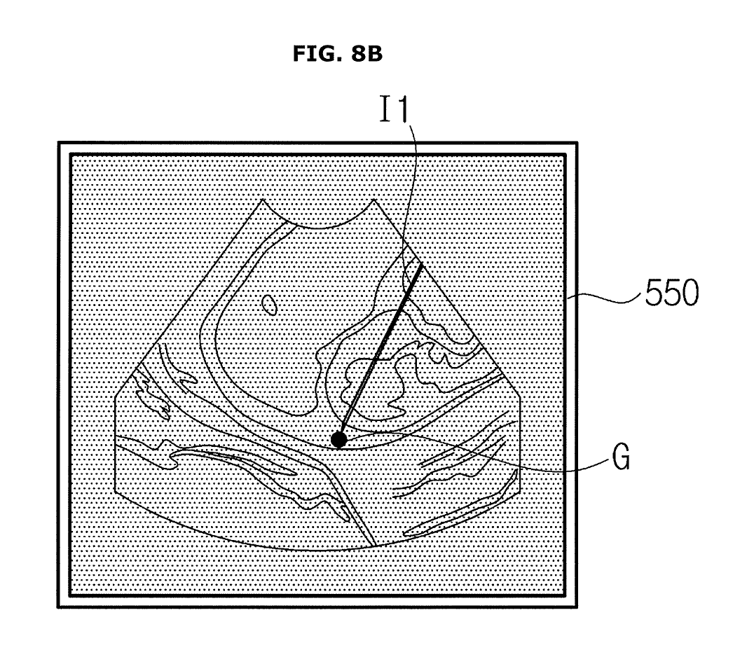

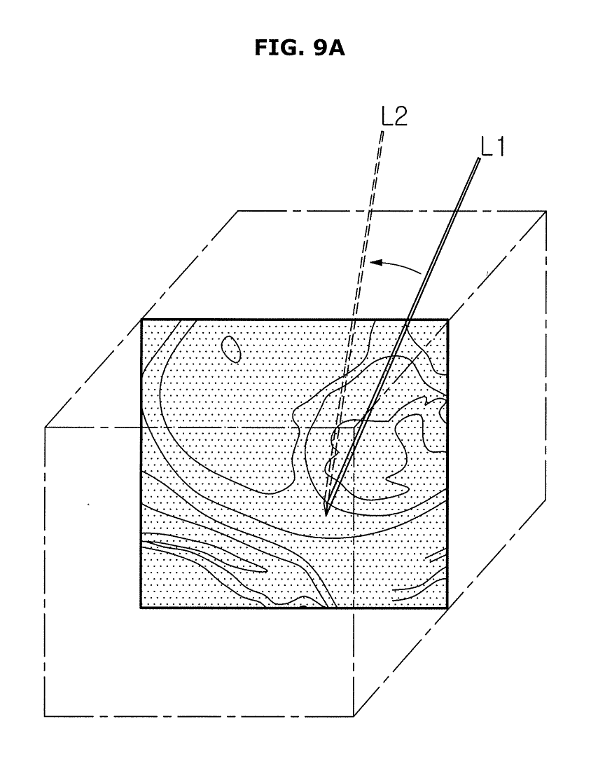

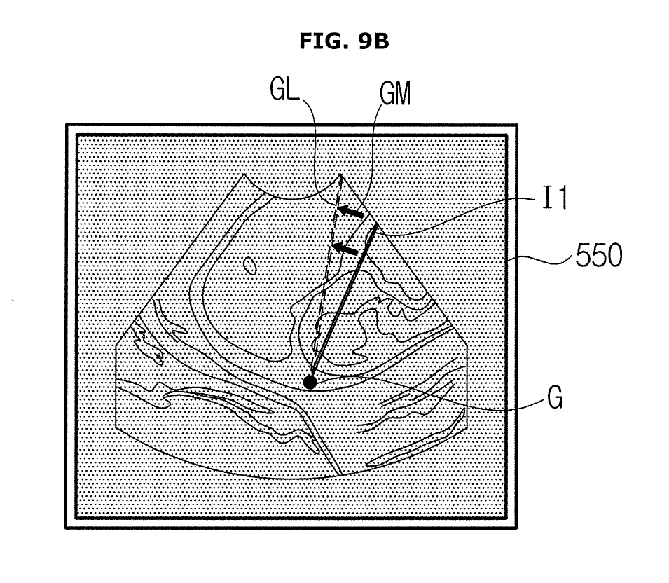

FIGS. 8A, 8B, 9A, and 9B are diagrams for describing operations of guiding a position of a needle N according to an embodiment.

FIGS. 8A and 8B are diagrams illustrating an image 11 of the needle N at an initial time point according to an embodiment.

Referring to FIG. 8A, the controller 500 indicates an initial position L1 of the needle N inserted into the object by the user.

Referring to FIG. 8B, the controller 500 may display the initial position 11 of the needle N on the display device 550. Methods of marking the initial position 11 of the needle N are not particularly limited. The user may set a target point G via the input device. The controller 500 of the ultrasound imaging apparatus may set the target point G based on previously stored information. Also, the initial position 11 may be displayed together with the target point G or separately. Since the operation of identifying the cross-sectional image including the needle N performed by the controller has been described above, detailed descriptions thereof will not be repeated.

FIGS. 9A and 9B are diagrams illustrating a guide line and a guide marker to guide the needle N according to an embodiment.

FIG. 9A is a diagram conceptually illustrating a needle inserted into an object by a user. FIG. 9B is a diagram illustrating an image of the needle displayed on the display device 550.

Referring to FIG. 9B, a guide line GL and a guide marker GM to change the initial position L1 of the needle N illustrated in FIG. 8B. When an initial insertion point of the needle N is determined, the controller 500 may display a path of the needle from the insertion point to the target point G, i.e., the guide line GL. In addition, the controller 500 may derive a difference between the positions L1 and L2 of the needle N controlled by the user and the guide line GL. In addition, the controller 500 may generate the guide marker GM to minimize the difference. According to an embodiment, the guide marker GM may be formed as an arrow. In addition, the controller 500 may control the guide marker GM to be located at a position to coincide a current position of the needle N with a previously formed guide line GL. Since the position of the needle N determined, by the controller 500, as an appropriate position to diagnose the object is different from the initial position 11 of the needle N in FIG. 9B, the controller 500 may control the guide marker GM to be displayed at the corresponding position to coincide the position of the needle N with the guide line GL. In addition, position change of the needle N (from L1 to L2) may be performed on the same cross-section. However, the embodiment is not limited thereto and the position change of the needle N may also include a rotation position change based on an angle of approach of the needle N.

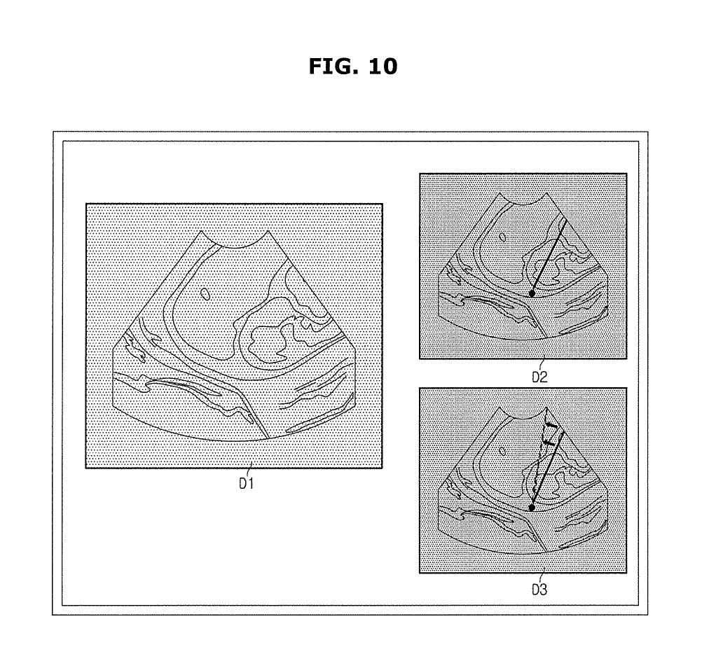

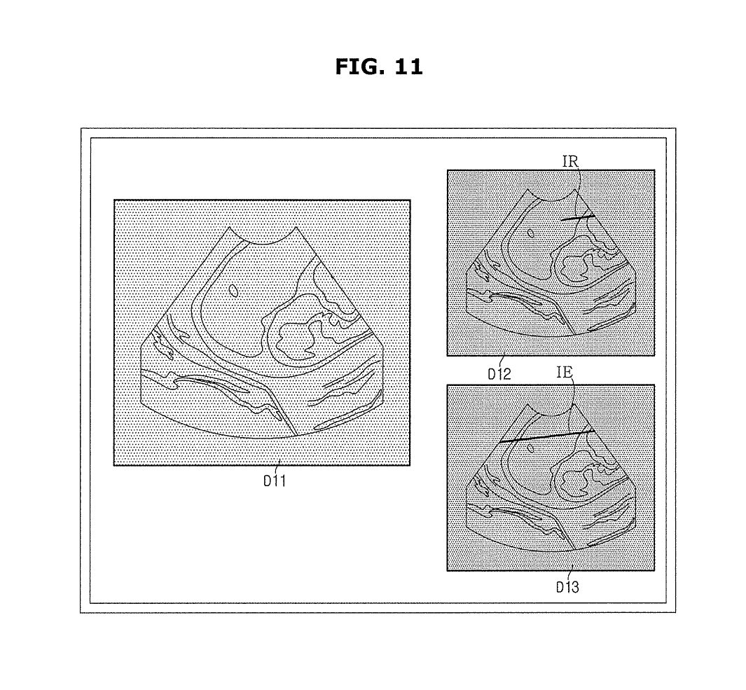

FIGS. 10 and 11 are diagrams illustrating operations of outputting an ultrasound image and an image to guide the position of the needle N according to an embodiment.

Referring to FIG. 10, the display device 550 according to an embodiment displays an ultrasound image D1, a cross-sectional image D2 indicating a current position of the needle N, and another cross-sectional image D3 including a guide line. Since an image for diagnosing the object by the user using the ultrasound probe and an image including the needle N are simultaneously displayed, the controller 500 may display the current position of the needle N inside the object allowing the user to identify the position. Also, a guide line or a guide marker to guide the needle N may be superimposed on the image, if required. Meanwhile, a process of identifying a cross-sectional image including the needle N among the cross-sectional images constituting the ultrasound image may be performed as described above for this operation.

Referring to FIG. 11, the display device 550 displays an ultrasound image D11, a cross-sectional image D12 indicating a current position IR of the needle N, and another cross-sectional image D13 showing information on a predicted position IE of the needle N.

The information on the predicted position may be derived based on position information from a point of time in the past to a current point of time at which measurement is performed. The controller 500 may derive position information of the needle N after the current point of time based on position information of the needle N from a point of time in the past to the current point of time or motion of the needle acquired by the sensing device. For example, in the case where the needle N is approaching the object, the controller 500 may derive information on the predicted position of the needle N after the current point of time based on current speed and angle of approach of the needle N. In addition, since the controller 500 provides the user with the information on the predicted position together with information on the current position of the needle N, the user may intuitively recognize the position of the needle N to be located when moving the needle N under the current conditions.

Meanwhile, the images illustrated in FIGS. 10 and 11 are merely examples of image arrangement according to the present disclosure and examples of outputting images to the display device 550 to display the position of the needle N or guide the needle N are not limited thereto.

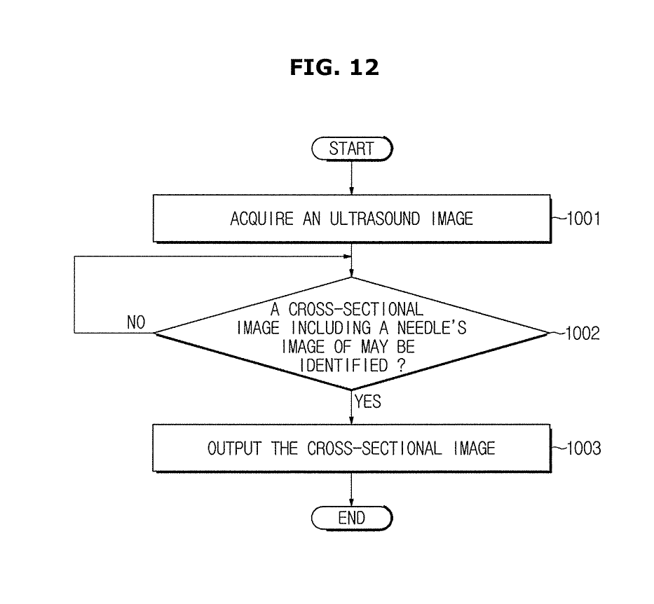

FIGS. 12 to 14 are flowcharts according to an embodiment.

Referring to FIG. 12, the ultrasound imaging apparatus may acquire an ultrasound image (1001). Among cross-sectional images constituting the acquired ultrasound image, a cross-sectional image including a needle's image of may be identified (1002). The cross-sectional image including the needle's image may be output (1003).

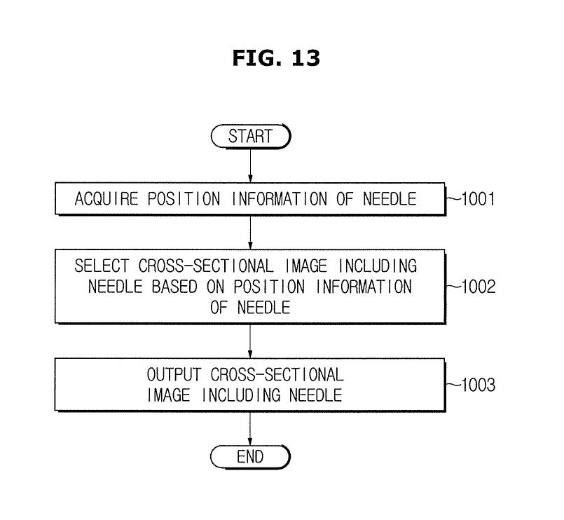

Referring to FIG. 13, the sensing device may acquire position information of the needle N (1011). The controller 500 may select a cross-sectional image including the needle N based on the position information of the needle N (1012). The controller 500 may output the selected cross-sectional image (1013).

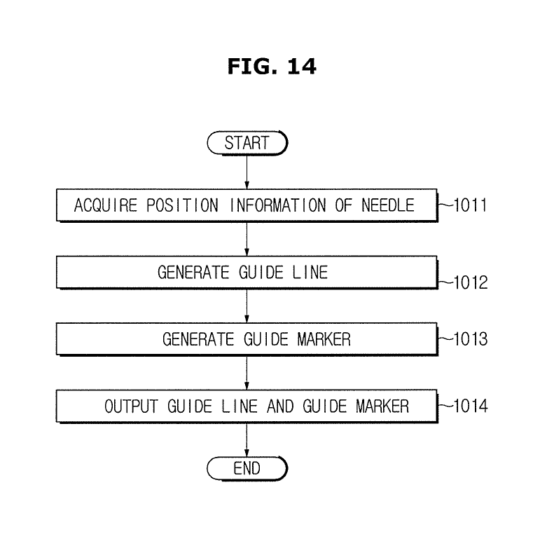

Referring to FIG. 14, a cross-sectional image including a needle's image may be selected based on the aforementioned method (1021). A guide line may be generated based on the position of the needle N (1022). In addition, the controller 500 may generate a guide marker to coincide a current position of the needle N with the guide line (1023). The controller 500 may output the generated guide line and guide marker to the display device 550 (1024).

Meanwhile, the aforementioned embodiments may be embodied in the form of a recording medium storing instructions executable by a computer. The instructions may be stored in the form of program codes and perform the operation of the disclosed embodiments by creating a program module when executed by a processor. The recording medium may be embodied as a computer readable recording medium.

The computer readable recording medium includes all types of recording media that store instructions readable by a computer such as read only memory (ROM), random access memory (RAM), magnetic tape, magnetic disc, flash memory, and optical data storage device.

Although a few embodiments of the present disclosure have been shown and described, it would be appreciated by those skilled in the art that changes may be made in these embodiments without departing from the principles and spirit of the disclosure, the scope of which is defined in the claims and their equivalents.

What is claimed is:

1. An ultrasound imaging apparatus comprising:
a display device;
a probe configured to acquire an ultrasound image by emitting ultrasound to a surface of an object; and
a controller configured to determine whether or not an image of a needle is comprised in at least one cross-sectional image constituting the ultrasound image of the object, and output the at least one cross-sectional image comprising the needle's image to the display device upon determination that the at least one cross-sectional image comprise the needle's image.

2. The ultrasound imaging apparatus of claim 1, wherein when a position of the needle is changed, the controller outputs the at least one cross-sectional image comprising the needle's image corresponding to a changed position of the needle to the display device.

3. The ultrasound imaging apparatus of claim 1, wherein the controller outputs the at least one cross-sectional image comprising the needle's image to the display device in real time.

4. The ultrasound imaging apparatus of claim 1, wherein when the needle is inserted into the object, the controller generates a guide line from an insertion point of the needle to a predetermined target point and outputs the guide line to the display device.

5. The ultrasound imaging apparatus of claim 3, wherein the controller derives a difference between the needle's image and the guide line, generates a guide marker based on a position of the guide line by using the needle's image as a reference, and outputs the guide marker to the display device.

6. The ultrasound imaging apparatus of claim 4, wherein the controller derives a guide marker based on a relationship between an extended line of the needle's image comprised in the at least one cross-sectional image and the position of the predetermined target point, and outputs the guide marker to the display device.

7. The ultrasound imaging apparatus of claim 1, wherein the controller tracks a position of the needle's image comprised in the at least one cross-sectional image in real time and outputs the at least one cross-sectional image corresponding to the needle's image to the display device.

8. The ultrasound imaging apparatus of claim 1, wherein the controller derives a predicted position of the needle after a current point of time based on positions of the needle's image from a point of time in the past to the current point of time,
derives the at least one cross-sectional image comprising the predicted position of the needle, and
outputs the at least one cross-sectional image to the display device.

9. The ultrasound imaging apparatus of claim 1, wherein when the needle is inserted into the object, the controller controls the ultrasound image of the object comprising at least one cross-sectional image comprising the needle's image to be output to the display device.

10. The ultrasound imaging apparatus of claim 1, further comprising a sensing device configured to acquire position information of the needle, wherein the controller derives the needle's image comprised in the at least one cross-sectional image based on the position information of the needle.

11. The ultrasound imaging apparatus of claim 1, wherein the ultrasound probe comprises at least one of a matrix probe and a three-dimensional (3D) probe.

12. A method of controlling an ultrasound imaging apparatus, the method comprising:
acquiring an ultrasound image by emitting ultrasound to a surface of an object,
determining whether or not an image of a needle is comprised in at least one cross-sectional image constituting the ultrasound image of the ultrasound image, and
outputting the at least one cross-sectional image comprising the needle's image to a display device upon determination that the needle's image is comprised in at least one cross-section image.

13. The method of claim 12, further comprising outputting the at least one cross-sectional image comprising the needle's image corresponding to a changed position of the needle to the display device when a position of the needle is changed.

14. The method of claim 12, wherein the outputting of the at least one cross-sectional image comprises outputting the at least one cross-sectional image comprising the needle's image in real time.

15. The method of claim 12, further comprising generating a guide line from an insertion point of the needle to a predetermined target point when the needle is inserted into the object.

16. The method of claim 15, further comprising deriving a difference between the needle's image and the guide line, and
generating a guide marker based on a position of the guide line by using the needle's image as a reference,
wherein the outputting of the at least one cross-sectional image further comprises outputting the guide marker to the display device.

17. The method of claim 16, wherein the generating of a guide marker comprises deriving a guide marker based on a relationship between an extended line of the needle's image comprised in the at least one cross-sectional image and a predetermined target point.

18. The method of claim 12, further comprising tracking a position of the needle's image comprised in the at least one cross-sectional image in real time.

19. The method of claim 12, further comprising deriving a predicted position of the needle after a current point of time based on positions of the needle's image from a point of time in the past to the current point of time, and deriving the at least one cross-sectional image comprising the predicted position of the needle.

20. The method of claim 12, wherein the outputting of the at least one cross-sectional image further comprises outputting an ultrasound image of the object comprising at least one cross-sectional image having the needle's image when the needle is inserted into the object.
